(19) United States
(12) Patent Application Publication
(10) Pub. No.: US 2016/0331720 A1
(43) Pub. Date: Nov. 17, 2016

(54) SCALEABLE CANNABINOID TREATMENT REGIMINE AND MEDICINAL FORMULATIONS

(71) Applicant: Andrew David Hospodor, Santa Cruz, CA (US)

(72) Inventor: Andrew David Hospodor, Santa Cruz, CA (US)

(21) Appl. No.: 14/714,222

(22) Filed: May 15, 2015

(51) Int. Cl. A61K 31/352; A61K 45/06; A61K 31/05

(52) U.S. Cl. CPC A61K 31/352; A61K 31/05; A61K 45/06

(57) ABSTRACT

The present invention administers a cannabinoid medication to a patient followed by observing one or more physiological responses in the patient during a first period of time when treating a medical condition. The cannabinoid medication may include one or more specific types of cannabinoids at an initial dosage level. In certain instances the cannabinoid dosage level in incremented over the first period of time. The dose of a cannabinoid corresponds to a mass of one or more cannabinoid types administered to the patient over a period of time, typically over a day. The cannabinoid treatment may be combined with one or more other methods for treating cancer, such as administering a chemotherapy agent, a KRAS inhibitor, or a CTLA-4 antigen. The present invention also includes a formulation of cannabinoid medications that span one or more ranges of milligram dosages of one or more cannabinoids in a set of formulary drugs.

### BACKGROUND OF THE INVENTION

1. Field of the Invention

The presently claimed invention relates to treating a cancer patient. More specifically the presently claimed invention relates to the administration of cannabinoids as a medicinal treatment to a cancer patient.

2. Descriptions of the Related Art

Many medical schools teach that that metastases are clonal of a primary tumor. However, aggressive adeno-carcinoma type cancers appear to constantly mutate due to miss-correction of the DNA. Pancreatic cancer is one of the deadliest forms of adeno-carcinoma, with patient survival measured in months. When treating pancreatic cancer, doctors expect chemotherapy agents to quell metastases, and this is simply not correct. Although they can originate from the primary tumor, metastases may be very different from the primary tumor. Furthermore, pancreatic primary tumor rarely kills the host. Instead, metastases of the cancer interfere with other endocrine organs, the intestinal tract and even the lungs. Certain forms of cancer overexpress numerous cannabinoid receptors, and Donadelli reported that the CannabinoidReceptor1 (CB1) ligand Rimonabant (SR141716, e.g. “SR1”) shrunk pancreatic cancer cells in mice. Adenocarcinoma is known to have many CB1 receptors, and THC is known to bind to CB1 receptors (i.e. THC is a CB1 ligand). Donadelli also discusses that cannabinoids when combined with Gemcitabine acts as autophagy and induces apoptosis in pancreatic cancer cells.

Chemotherapy is a known treatment regimen used to treat cancer. One commonly used chemotherapy agent is Gemcitabine. “Gemcitabine is a member of a group of chemotherapy drugs known as anti-metabolites. It prevents cells from making DNA, which stops cell growth and causes the cells to die.” (Quote from cancer.org). Antimetabolites are very similar to normal substances within the cell, when cells incorporate an antimetabolite into their cellular metabolism, the cells cannot function properly and are unable to divide. Since antimetabolites are cell-cycle specific they attack cells at very specific phases in their life cycle. Antimetabolites are classified according to the substances with which they interfere, i.e. they are antagonize or inhibit folic acid, pyrimidine, purine, and adenosine deaminase. Examples include: Folic acid antagonist: Methotrexate I Pyrimidine antagonist: 5-Fluorouracil, Foxuridine, Cytarabine, Capecitabine, and Gemzar I Purine antagonist: 6-Mercaptopurine and 6-Thioguanine I Adenosine deaminase inhibitor: Cladribine, Fludarabine and Pentostatin.

Antimetabolites, like many chemotherapy agents are toxic to all cells, not just cancer cells. Because cancer cells grow much faster than normal cells, they are disproportionately affected by the chemotherapy.

A proposed method to treat adenocarcinoma involves administering a substance that interferes with a Kirsten rat sarcoma oncogene homolog (i.e. a KRAS inhibitor) from the mammalian ras gene family. The KRAS gene is known to produce a protein that is commonly referred to as K-Ras that is known to produce proteins that accelerate growth. Mutations in the KRAS gene can result in normal cells dividing uncontrollably with an increased probability of turning cancerous. Recently a KRAS inhibitor was identified as the molecule SML-8-73-1 by the University of Texas Southwester Cancer Research Center.

Currently physicians cannot prescribe and medical dispensaries do not have cannabinoid medications that span a range of formulations. Because of this a patient cannot currently be provided with cannabinoid based medications according to a treatment regimen where the dosage of specific cannabinoids may be easily varied over time. Formulations of cannabinoid based medications that span a range of cannabinoid dosages are needed for treating cancer or other ailments that may easily be administered to a patient.

Human patents administered high dosage levels of THC, such as Marinol or Drobinol, often experience psychoactive side effects of the drug to an extent that may cause the patient to avoid the medication. Because of this a regimen that mitigates THC while allowing the patient to adjust to side effects of the cannabinoid is needed.

Conventional cancer treatments do not attack cancer using multiple pathways. What is needed is a regimen that treats cancer by attacking cancer in a plurality of different ways. Such a treatment could exploit the pathways associated with CB1 ligands, Antimetabolites and KRAS.

### SUMMARY OF THE PRESENTLY CLAIMED INVENTION

An embodiment of the presently claimed invention administers a cannabinoid medication to a patient followed by observing one or more physiological responses in the patient during a first period of time. The cannabinoid medication may include one or more specific types of cannabinoids at an initial dosage level. In certain instances the cannabinoid dosage level is incremented over the first period of time. The dose of a cannabinoid corresponds to a weight of one or more cannabinoid types administered to the patient over a period of time, typically over a day. The presently claimed invention may be used to treat various aliments by administering of cannabinoids over time.

The cannabinoid treatment may be combined with one or more other methods for treating cancer, such as administering a chemotherapy agent and/or a KRAS inhibitor. When the cannabinoid medication is administered at a first dosage level that is subsequently increased as opposed to starting the patient at a high dosage level, the patient's body is given an opportunity to adapt to or to gain tolerance to side effects that may be associated with a cannabinoid. Furthermore, since lower dosages of certain cannabinoids may mitigate anxiety, increase appetite, reduce swelling, block pain, and reduce nausea the present invention allows a patient to receive immediate benefits while minimizing side effects that may be associated with consuming cannabinoids. When a cannabinoid medication regimen is begun before or when a chemotherapy agent is administered, the present invention may help mitigate side effects (nausea, for example) associated with the chemotherapy agent. The presently claimed invention may also utilize cannabinoid medications that may be administered in a pill, capsule, or suppository form according to formulations that span one or more varying ranges of cannabinoid dosages. By providing formulations of cannabinoid dosages over a varying range, a practitioner may easily change and monitor cannabinoid dosages administered to a patient over time. Since, in one instance, a practitioner may be treating cancer and, in another instance, be treating another ailment of the patient, a range of different cannabinoid formulations in a pill or capsule provide the practitioner with great flexibility when changing a cannabinoid treatment regimen over time. Formulary medications of the presently claimed invention may scale cannabinoid levels in a series of steps where cannabinoid levels increase or decrease according to at least an exponential function from one pill to another.

### BRIEF DESCRIPTION OF THE DRAWINGS

FIG. 1 illustrates a graph of data observed by Donadelli when treating pancreatic cancer in mice.

FIG. 2 illustrates the administration of varying amounts of THC and CBD over time and illustrates physiological factors measured when treating pancreatic cancer in human Patient-0.

FIG. 3 illustrates an exemplary methodology consistent with the presently claimed invention.

### DETAILED DESCRIPTION

An embodiment of the presently claimed invention administers a cannabinoid medication to a patient followed by observing one or more physiological responses in the patient during a first period of time. The cannabinoid medication may include one or more specific types of cannabinoids at an initial dosage level. In certain instances the cannabinoid dosage level is incremented over the first period of time. The dose of a cannabinoid corresponds to a mass, in grams, of one or more cannabinoid types administered to the patient over a period of time, typically over a day. When treating cancer, the cannabinoid treatment may be combined with one or more other methods for treating cancer, such as, administering a chemotherapy agent, a KRAS inhibitor, or both. When treating other ailments, a practitioner may select a cannabinoid formulation from a plurality of cannabinoid medications according to a treatment regime.

The present invention for treating cancer attacks cancer in several different ways: 1. Trigger autophagy/apoptosis in cancer cells through a Reactive Oxgyen Species (ROS) mediated mechanism using cannabinoids; 2. Modulate DNA mis-repair with an antimetabolite chemotherapy agent; 3. Slow the grow Suppress the KRAS gene with a KRAS inhibitor; 4. Target mutant antigens with checkpoint blockade immunotherapy. Donadelli has already demonstrated 1 in Italy by reducing pancreatic cancer tumors in mice after administering high dosages of CB1 ligands such as SR1. Donadelli, however, never adapted this treatment for human trials, never combined tetrahydrocannabinol (THC) with cannabidiol (CBD), never considered treating cancer with a KRAS inhibitor or with a checkpoint blockade immunotherapy. Checkpoint blockade immunotherapy is discussed by D. R. Leach, et al in a document entitled: “Enhancement of Antitumor Immunity by CTLA-4 Blockade,” Science, 22 Mar. 1996.

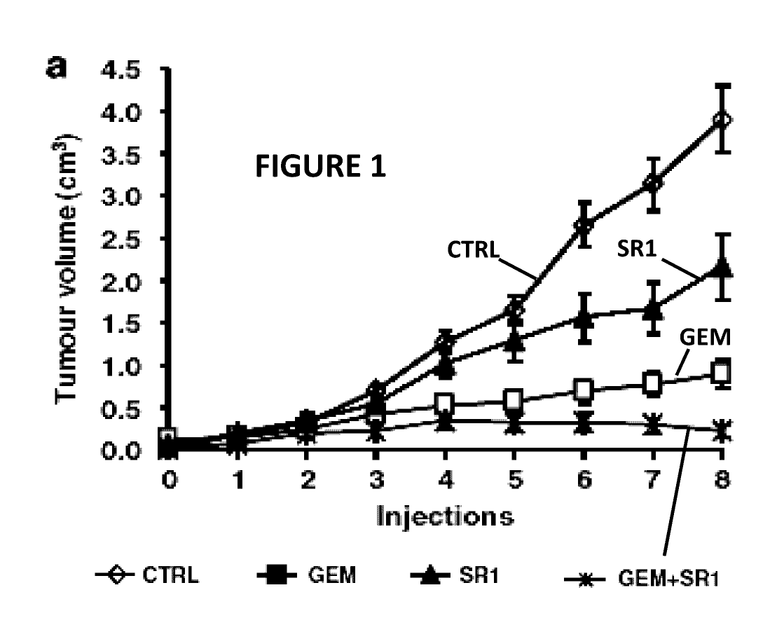

FIG. 1 illustrates a graph of data observed by Donadelli when treating pancreatic cancer in mice. FIG. 1 includes four separate experiments where the volumes of tumors were measured over time while administering medications according to four different treatment regimens. In a first experiment a control group received no medication. Note that tumor volume increased most dramatically in the control group curve CRTL. Note also that when CB1 ligand SR1 was administered in a number of injections the tumor volume grew at a slower rate in the SR1 curve as compared to the control group curve CRTL. FIG. 1 also shows that when Gemcitabine was injected over time, tumor volume grew at even a slower rate as indicated by the GEM curve including lower tumor volumes than the SR1 curve. Notice also that while the rate of growth of pancreatic cancer was slowed when SR1 or Gemcitabine were administered to the mice, the volume of the cancer tumors continued to increase throughout the experiment in the SR1 and in the GEM curves. The fourth curve GEM+SR1 in FIG. 1 illustrates data taken by Donadelli when both the SR1 CB1 ligand and Gemcitabine were combined. In the GEM+SR1 curve the tumor volume peaked by the fourth injection. The GEM+SR1 curve also shows that tumor volume was markedly reduced by the eighth injection.

Embodiments of the present invention may first administer an initial cannabinoid dosage to a patient while one or more physiological effects of the cannabinoid may be monitored. The present invention may also increase the dosage of cannabinoids over time while monitoring one or more pathological factors until a pathological effect has been observed.

The one or more physiological effects may also be monitored over the course of treatment. In the instance where a physiological effect that was initially observed is determined to no longer be observed or is observed at a reduced level after a cannabinoid administration, a determination may be made that the patient's body is adapting to the presence of the cannabinoids.

Subsequently, a practitioner may identify that a patient may have stopped taking the cannabinoid medication when a physiological effect is once again observed after the patient consumes a cannabinoid medication. In the instance where a practitioner identifies that a patent may have stopped taking the cannabinoid medication, the practitioner may have the patient watched more closely. Reasons why a patient may stop taking a medication include: forgetfulness, distractions, and confusion. In certain instances a patient may forget because of their weakened condition, in another instance the patient be distracted by thinking about their condition, or in yet another instance the patient may be confused when they lose track of passing time.

Thus, by monitoring that a cannabinoid related physiological effect was first observed, was subsequently observed to abate, and then was observed to return after cannabinoids were administered may be used to identify that a patient requires additional assistance in maintaining a recommended treatment regimen.

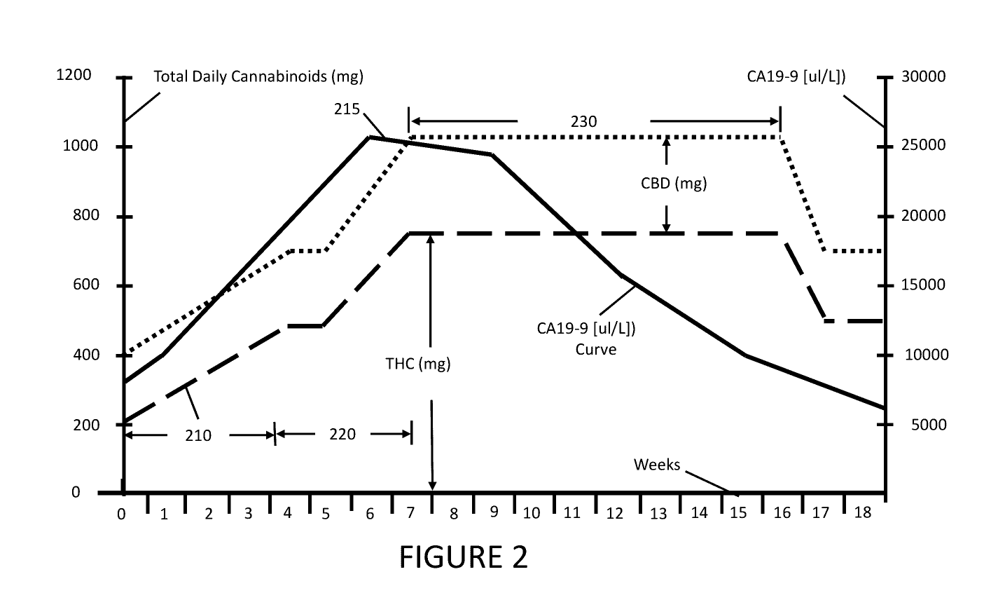

The Applicant notes that both physiological effects and pathological factors were observed in a human patient suffering from stage 4 pancreatic cancer. The human patient, herein, referred to a “Patient-0” was treated according to the treatment mythology illustrated in FIG. 2. Patient-0 received varying amounts of THC and CBD over time and FIG. 2 illustrates physiological factors measured when treating pancreatic. The graph of FIG. 2 includes a first vertical axis of total daily cannabinoids in milligrams (mg), a second vertical axis of bio-marker CA19-9 measured in micro-liters per liter (ul/L), and a horizontal axis of time in weeks. Cannabinoid dosages, including THC and CBD, are started and increased over a first period of time 210. The curve shown with the large dashes shows a measure of THC being administered to the patient every day. The curve shown with the small dashed shows a measure of THC (mg) plus CBD (mg) being administered to the patient every day. Note that a measure of CBD being administered may be determined by subtracting the milligram (mg) dosage indicated by the small dashed curve minus the mg dosage shown by the large dashed curve. For example at time zero an initial total cannabinoid dosage of 400 mg/day was administered to the patient. Of that 400 mg/day 200 mg was THC, so the mg/day dosage of CBD was 400 mg-200 mg=200 mg. Patient-0 also received 155 mg Gemcitabine in 250 ml saline solution on a schedule of two weeks on/one week off. Patient-0 also received cannabinoid treatments in capsule form according to the graph of FIG. 2. After 7 days of cannabinoid treatment Patient-0 stopped presenting symptoms of cannabis used, such as red eyes, slurred speech, and clumsiness. After 10 day Patient-0 reported no longer feeling euphoric effects of cannabinoid use (i.e. the patient no longer felt “high” or “stoned”). Patient-0, therefore, adapted to the cannabinoids because Patient-0 stopped exhibiting various physiological effects after 7 to 10 days on the cannabinoid treatment regimen.

Cannabinoid dosages are then increased again in a second period of time 220 until levels of blood bio-marker C19-9 are observed reducing 215, then cannabinoid dosages are maintained over a third period of time 230 while the blood bio-marker C19-9 continues to reduce. Notice also that relative mg dosages of THC versus CBD may also be varied over time where an amount of THC versus CBD may be increased at a greater rate relative over time or visa-versa. FIG. 2 also shows cannabinoid dosages being reduced after the third period of time 230. The size of Patient-0's pancreatic tumors where also observed to reduce over the course of the treatment regimens administered to Patient-0.

Parametric data relating to the treatment of Patient-0 was presented in May 2014 to the American Association of Cancer Researchers at a special conference on pancreatic cancer in Louisiana. This data was also provided to members of the United States Congress when representatives of PanCan.org visited Washington D.C.

High doses of THC may cause cancer tumors to shrink by inducing the death of cancer cells by the mechanisms of autophagy and apoptosis. The cancer may be attacked by promoting the digestion of cellular constituents by enzymes of the same cell (autophagy), and by accelerating a genetically determined process of cell self-destruction that may be marked by the fragmentation of nuclear DNA (apoptosis).

Since apoptosis may be activated either by the presence of a stimulus or by the removal of a stimulus or suppressing agent, different cannabinoid types may activate apoptosis in different types of cancer cells. The present invention may also include identifying a type of cancer by identifying its genome and by identifying whether the cancer includes specific types of cannabinoid receptors. This process may also include determining how many cannabinoid receptors are included in a cancer cell relative to the number of cannabinoid receptors that are included in a normal cell. Since forms of pancreatic cancer have been found to have a 100× more CB1 receptors than normal cells, and since THC bonds with the CB1 receptor, a patient consuming high levels of THC may saturate the pancreatic cancer cells with THC. The treatment regimen may also include administering combinations of THC, CBD and other cannabinoids for various reasons. For example, by administering CBD a patient should receive anxiety relief.

By administrating specific cannabinoids over a first period of time, a patient receives anxiety relief and is allowed to acclimate to cannabinoids while the cannabinoids begin interfering with metastatic processes that are related to the spread of cancer. Cannabinoid dosages may be increased in one or more steps over a period of time.

When administering cannabinoids the method for treating cancer may begin by observing a physiological response in a person after administrating one or more cannabinoid medications according to a first treatment dosage regimen. That regimen may include administering more specific types of cannabinoids over a first period of time in one or more discrete administrations. Physiological responses observed may include red eyes, slurred speech, forgetfulness, clumsiness, and lack of attention. Other physiological responses observed may include muscle relaxation, reduced sensitivity to pain, reduced swelling/inflammation, and increased mobility.

The method for treating cancer may also include administrating one or more different cannabinoid medications according to a second treatment dosage regime over a second period of time while monitoring one or more pathological factors. The one or more pathological factors monitored during the second period of time may include monitoring the size of a tumor or monitoring a bio-marker in the blood of a patient. When treating some forms of adenocarcinoma the monitored bio-marker may be C19-9. When a desirable pathological effect is observed a practitioner could conclude that the treatment regime is benefiting the patient. Alternatively both physiological effects and pathological factors may be observed during the first period of time until a desirable pathological effect is observed. After a desired pathological effect has been observed cannabinoid dosages may be maintained or be increased again while monitoring the pathological factors. When a physiological response is observed to abate after administering a cannabinoid, a practitioner may determine that the patient is adapting to the cannabinoids that they have been administered.

The method of the invention may also include administrating a third treatment cannabinoid dosage over a third period of time, and a fourth cannabinoid dosage over a fourth period of time. The cannabinoid dosage administrated over the fourth period of time may be a reduced dosage as compared to dosage administered over the third period of time.

A discussed above a cannabinoid regimen for treating cancer that has been adapted to humans may include increasing cannabinoid dosages over time and include a chemotherapy agent, a KRAS inhibitor, or both.

The present invention also includes a plurality of different formulations of cannabinoid medications that include different proportions/ratios or relative milligram dosages of different cannabinoids. The different formations may be provided to a practitioner in pill or capsule form. The formulations may also allow the practitioner to change dosages by administering a different pill or capsule from a plurality of different formulations. An exemplary table of different formulations consistent with the present invention is shown in table 1. The cannabinoid formulations of the present invention may be used to treat cancer or other ailments including, yet not limited to Parkinson's disease, epilepsy, or neuropathic pain. Table 1 includes low THC, balanced THC-CBD, and low CBD medication formulations. Note how the formulations scale in a series of steps that are similar to an exaggerated exponential. For example, an exponential scales [1, 2, 4, 8, 16] where entries in the table scale [1, 2, 5, 10, 20]. Other exemplary formulations include [2, 4, 10, 20, 40], [10, 5, 2, 1, 0.5], [50, 125, 250, 500, 1250], and [100, 200, 500, 1000, 2000].

Note that each of the formulations in table 1 include two different cannabinoids. While TCH and CBD are illustrated, other cannabinoids may also be included in a formulation consistent with the present invention. Cannabigerol (CBG), for example, may be included in a formulation. By providing health practitioners with cannabinoid medications that scale, a practitioner may adjust dosages provided to a patient as desired. Also by providing two or more different cannabinoids a practitioner may select a formulation based on a treatment strategy. When treating epilepsy or spasticity disorders, a high CBD formulation may be selected. When treating forms of cancer with a high number of CB1 receptors, a high THC formulation may be selected. In other instances a balanced cannabinoid medication may be administered.

TABLE 1
Dosage levels of cannabinoids

| Low THC | | Balanced THC-CBD | | Low CBD | |
|---|---|---|---|---|---|
| 0.5 | 10 | 10 | 10 | 10 | 1 |
| 1 | 20 | 20 | 20 | 20 | 2 |
| 2 | 50 | 50 | 50 | 50 | 5 |
| 5 | 100 | 100 | 100 | 100 | 10 |
| 10 | 200 | 200 | 200 | 200 | 20 |
| THC mg | CBD mg | THC mg | CBD mg | THC mg | CBD mg |

The embodiment of the present invention that combines a cannabinoid treatment, chemotherapy, and a KRAS inhibitor may prove to be the most effective way to treat certain forms of cancer.

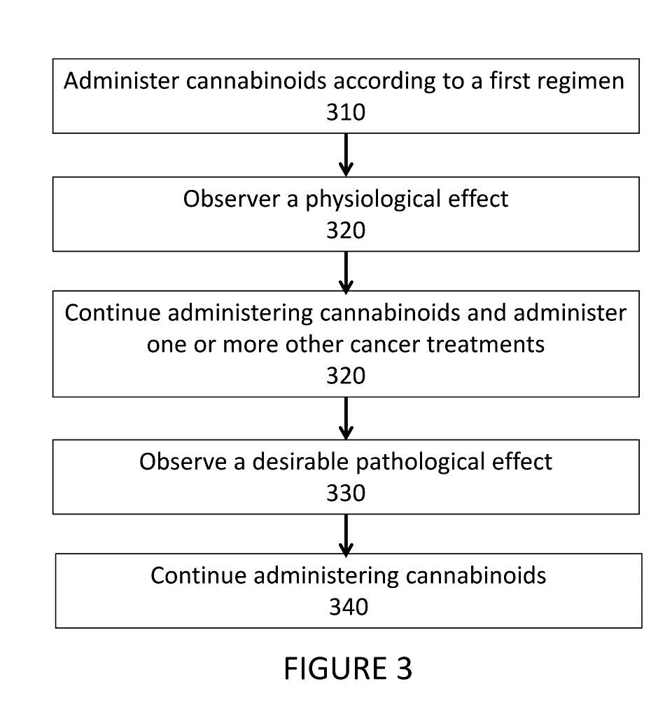

FIG. 3 illustrates an exemplary methodology consistent with the presently claimed invention. In step 310 cannabinoids are administered according to a first treatment regimen. The first treatment regimen may include increasing mg dosages of cannabinoids over time. In step 320 a physiological effect of the person is observed. Next in step 320 the administration of cannabinoids may be continued and one or more other cancer treatment methods may be administered to the patient. Step 320 may also include increasing cannabinoid dosage levels administered to the patient on a daily basis. Then in step 330 a pathological effect may be observed. Finally in step 340 the administration of cannabinoids may be continued.

Note that the present invention may begin by administrating cannabinoids to a patient followed by administering a second cancer fighting methodology. The present invention may also begin by administering cannabinoids combined with one or more other methods for fighting cancer. When concerned with mitigating the effects of nausea induced by chemotherapy a chemotherapy treatment may be preceded by one or more cannabinoid treatments. Furthermore, cannabinoids may be administered after chemotherapy treatments or KRAS treatments have been terminated. The method may also include administering an immunotherapy agent that stimulates an increased immune response. Exemplary immunotherapy agents that may be administered include, yet are not limited to administering: an cytotoxic T-lymphocyte antigen 4 (CTLA-4) blocking treatment, an agent that affects the programmed cell death 1 receptor (PD-1) on T cells, a vaccine, a bacteria (dead or alive), or by administering an adoptive T cell transfer regimen. An adoptive T cell transfer regimen includes removing T cells are removed from a patient, expanding the T cells in a laboratory, and re-infusing the expanded T cells into the patient.

In certain instances an anti-CTLA-4 or an anti-PD-1 immunotherapy agent may be administered. Since anti-CTLA-4 antibodies such as ipilimumab have been associated with the side effect of excessive inflammation leading to increased rates of colitis, dermatitis, and hepatitis combining and anti-CTLA-4 agent with an anti-inflammatory cannabinoid treatment. The combining an anti-CTLA agent with cannabinoids the negative side effects of drugs like ipilimumab may be mitigated. As such an immunotherapy may be administered before, after, or coincident with administering a cannabinoid regimen to a patient.

The method may also include administering an immunotherapy agent that stimulates an increased immune response. Exemplary immunotherapy agents that may be administered include, yet are not limited to administering: an cytotoxic T-lymphocyte antigen 4 (CTLA-4) blocking treatment, an agent that affects the programmed cell death 1 receptor (PD-1) on T cells, a vaccine, a bacteria (dead or alive), or by administering an adoptive T cell transfer regimen. An adoptive T cell transfer regimen includes removing T cells are removed from a patient, expanding the T cells in a laboratory, and re-infusing the expanded T cells into the patient.

In certain instances an anti-CTLA-4 or an anti-PD-1 immunotherapy agent may be administered. Since anti-CTLA-4 antibodies such as ipilimumab have been associated with the side effect of excessive inflammation leading to increased rates of colitis, dermatitis, and hepatitis combining and anti-CTLA-4 agent with an anti-inflammatory cannabinoid treatment. The combining an anti-CTLA agent with cannabinoids the negative side effects of drugs like ipilimumab may be mitigated. As such an immunotherapy agent such as CTLA-4 may be administered before or after administering a cannabinoid regimen to a patient.

Cannabinoids can be administered to a patient before, in combination with, or after other one or more other therapies.

1. A method for treating a medical condition, the method comprising:
administering one or more cannabinoids to a patient according to a first treatment regimen to a patient, wherein a dosage of at least one of the one or more cannabinoids are increased over a first period of time;
monitoring one or more pathological factors of the patient; and
observing a pathological event corresponding to the one or more pathological factors, wherein the observed pathological event indicates that the medical condition is improving.

2. The method of claim 1, further comprising maintaining administering the increased dosage of the one or more cannabinoids after the desirable pathological event has been observed.

3.-11. (canceled)

18. A formulation of matter comprising of a plurality of discrete cannabinoid formulations, wherein the plurality of discrete cannabinoid formulations change at least according to an exponential function in a series of steps, each of the discrete cannabinoid formulations formed as a dispensable unit of a plurality of dispensable units, and each of the dispensable units of the series of dispensable units including a first cannabinoid type of tetrahydrocannabinol (THC) and a second cannabinoid type of cannabidiol (CBD).

19. The formulation of matter of claim 18, wherein:
a weight of THC increases according to the at least exponential function and a weight of CBD decreases according to the at least exponential function in each step of the series of steps,
a first step of the series of steps that corresponds to a first dispensable unit formulation,
a second step of the series of steps that corresponds to a second dispensable unit formulation, and
a third step of the series of steps that corresponds to a third dispensable unit formulation.

20. The formulation of matter of claim 18 wherein:
a weight of THC and weight of CBD increases according to the at least exponential function in each step of the series of steps,
a first step of the series of steps that corresponds to a first dispensable unit formulation,
a second step of the series of steps that corresponds to a second dispensable unit formulation, and
a third step of the series of steps that corresponds to a third dispensable unit formulation.

21. The formulation of matter of claim 18, wherein:
a weight of THC increases according to the at least exponential function in the series of steps,
a first step of the series of steps that corresponds to a first dispensable unit formulation,
a second step of the series of steps that corresponds to a second dispensable unit formulation, and
a third step of the series of steps that corresponds to a third dispensable unit formulation.

22. The formulation of matter of claim 18, wherein:
a weight of CBD increases according to the at least exponential function and a weight of THC increases in the series of steps,
a first step of the series of steps that corresponds to a first dispensable unit formulation,
a second step of the series of steps that corresponds to a second dispensable unit formulation, and
a third step of the series of steps that corresponds to a third dispensable unit formulation.

23. The formulation of matter of claim 18, wherein each of the dispensable units of the plurality of dispensable units correspond to at least one of a pill, a capsule, or a suppository.

24. The formulation of matter of claim 18, wherein the series of steps corresponds to:
a first step of the series of steps that corresponds to a first dispensable unit formulation,
a second step of the series of steps that corresponds to a second dispensable unit formulation, and
a third step of the series of steps that corresponds to a third dispensable unit formulation, and
the first dispensable formulation, the second dispensable formulation, and the third dispensable formulation are each contained within a type of dispensable unit, and the type of dispensable units include at least one of a pill, a capsule, or a suppository.

25. The formulation of matter of claim 18, wherein:
a weight of CBD increases according to the at least exponential function and a weight of THC decreases according to the at least exponential function in each step of the series of steps,
a first step of the series of steps that corresponds to a first dispensable unit formulation,
a second step of the series of steps that corresponds to a second dispensable unit formulation, and
a third step of the series of steps that corresponds to a third dispensable unit formulation.

26. The formulation of matter of claim 18, wherein:
a weight of CBD increases according to the at least exponential function in the series of steps,
a first step of the series of steps that corresponds to a first dispensable unit formulation,
a second step of the series of steps that corresponds to a second dispensable unit formulation, and
a third step of the series of steps that corresponds to a third dispensable unit formulation.

27. The formulation of matter of claim 18, wherein:
a weight of CBD increases according to the at least exponential function and a weight of THC increases in the series of steps,
a first step of the series of steps that corresponds to a first dispensable unit formulation,
a second step of the series of steps that corresponds to a second dispensable unit formulation, and
a third step of the series of steps that corresponds to a third dispensable unit formulation.

28. The formulation of matter of claim 18, wherein a cancer patient consumes at least some of the dispensable units of the plurality of dispensable units as a cancer treatment.
